(19) United States
(12) Patent Application Publication
(10) Pub. No.: US 2006/0130244 A1
(43) Pub. Date: Jun. 22, 2006

(54) Aqueous composition for dyeing keratin fibres comprising a dye and a specific block copolymer

(76) Inventors: Franck Giroud, Clichy (FR); Lydia Panangatte, Poissy (FR); Claude Dubief, le Chesnay (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/285,189

(22) Filed: Nov. 23, 2005

Related U.S. Application Data
(60) Provisional application No. 60/635,961, filed on Dec. 15, 2004.

(30) Foreign Application Priority Data
Nov. 25, 2004 (FR) 04 52761

(51) Int. Cl. A61K 8/00

(52) U.S. Cl. 8/405

(57) ABSTRACT

The invention relates to a composition for dyeing keratin fibres, comprising a physiologically acceptable aqueous medium, comprising: at least one dye bearing at least one ionic group, said dye being present in an amount ranging from 0.001 to 5% by weight relative to the total weight of the composition; at least one block copolymer comprising at least one water-soluble nonionic block and at least one ionic block exhibiting charges opposite to that of the ionic charge borne by the dye.

This application claims benefit of U.S. Provisional Application No. 60/635,961, filed Dec. 15, 2004, the contents of which are incorporated herein by reference. This application also claims benefit of priority under 35 U.S.C. § 119 to French Patent Application No. 04 52761, filed Nov. 25, 2004, the contents of which are also incorporated by reference.

### TECHNICAL FIELD

The present invention relates to an aqueous composition for dyeing keratin fibres, in particular human keratin fibres, and more particularly the hair, which comprises at least one dye bearing an ionic charge, and a specific block copolymer.

The invention also relates to a process for dyeing keratin fibres, in particular human keratin fibres, and more particularly the hair, which uses said composition.

### STATE OF THE PRIOR ART

The dyestuffs industry markets a large number of molecules that find their use in various industrial fields. Among these molecules, some, which bear anionic groups such as a sulphonate or carboxylate group, or cationic groups such as primary, secondary and tertiary ammonium groups, are, because of their affinity with keratin and their results in dyeing, used in the formulation of hair dyeing products.

These molecules can be introduced into aqueous carriers, which, after application to the hair, will allow their transfer. In this case, it is desirable for the dyes to be and to remain soluble in the carriers in question, to be taken up rapidly onto the hair, but evenly whatever the sensitization thereof, and to resist repeated actions of washing and exposure to light.

These molecules can also be introduced into oxidation dyeing products in order to modulate the shades thereof. In this case, it is desirable for the dyes, in addition to the abovementioned properties, to exhibit a resistance to the alkaline oxidizing medium that characterizes these products.

At the current time, it is found that dyes bearing an anionic group or groups do not exhibit completely satisfactory properties.

Now, after lengthy research carried out on the question, the applicant has discovered that, surprisingly, the combination of a specific block copolymer with a dye bearing an ionic group makes it possible to increase the solubility in aqueous medium and/or the stability in oxidizing and reducing medium, and/or the uptake and/or the chromaticity on hair, of an ionic dye. The applicant has also discovered that such a combination facilitates unison, i.e. the homogeneous distribution of the coloration along the same keratin fibre and between keratin fibres. Furthermore, it is this discovery that forms the basis of the invention.

### DISCLOSURE OF THE INVENTION

Thus, the invention relates, in the first place, to a composition for dyeing keratin fibres, comprising a physiologically acceptable aqueous medium, comprising:
at least one dye bearing at least one ionic group, said dye being present in an amount ranging from 0.001 to 5% by weight relative to the total weight of the composition;
at least one block copolymer comprising at least one water-soluble nonionic block and at least one ionic block exhibiting charges opposite to that of the ionic charge borne by the dye.

According to the invention, the dyes can be chosen from acidic direct dyes and basic direct dyes.

It should be recalled that a direct dye is a coloured substance that does not require the use of an oxidizing agent in order to reveal its colour. In the remainder of this exposure, the terms “acidic dyes” and “basic dyes” will be used.

It is specified that the term “acidic dye” is generally intended to mean a dye containing at least one COOH, SO₃H, PO₃H, or PO₄H₂ group, it being possible for said groups to exist in the form of salts.

It is specified that the term “salts” is generally intended to mean salts of metals (for example, alkali metals or alkaline earth metals), salts of an organic amine that is optionally hydroxylated.

These dyes are also known as anionic dyes.

It is also specified that the term “basic dyes” is intended to mean a dye that has at least one group bearing a positive charge, such as an ammonium group or a quaternized nitrogen atom in a ring.

Direct dyes that have one or more optionally substituted amines not directly linked to an aromatic or heterocyclic ring can also be suitable in the present invention as a basic dye, although not preferably.

These dyes are also known as cationic dyes.

The acidic dyes that can be used in the context of this invention can be chosen from acidic nitro dyes, acidic azo dyes, acidic azine dyes, acidic triarylmethane dyes, acidic quinone dyes, acidic indo-amine dyes and acidic natural dyes, and mixtures thereof.

By way of illustration, mention may be made of the following dyes:
sodium salt of 2,4-dinitro-1-naphthol-7-sulphonic acid;
Acid Orange 3;
Acid Yellow 9/Food Yellow 2;
Direct Red 45/Food Red 13;
Acid Black 52;
Acid Yellow 36;
sodium salt of 1-hydroxy-2-(2′,4′-xylyl-5-sulphonatoazo)naphthalene-4-sulphonic acid/Food Red 1;
Acid Red 14/Food Red 3/Mordant Blue 79;
Acid Red 18;
Acid Brown 4;
Acid Orange 7/Pigment Orange 17/Solvent Orange 49;
Food Yellow 3/Pigment Yellow 104;
Acid Red 27/Food Red 9;
Acid Orange 10/Food Orange 4;
Acid Red 44;
Acid Red 33/Food Red 12;
sodium salt of 1-(3′-nitro-5′-sulpho-6′-oxophenylazo)-8-acetamidonaphthalene-3,6-disulphonic acid/Food Red 11;
sodium salt of 1-hydroxy-2-(2′-methylphenylazo)-8-acetamidonaphthalene-3,6-disulphonic acid/Acid Red 35;
Acid Violet 3;
Acid Violet 43;
Acid Red 35;
Acid Violet 7;
Acid Red 135;
Acid Yellow 27;
Acid Yellow 23/Food Yellow 4;
Acid Yellow 36;
4′-(sulphonato-2″,4″-dimethyl)bis-(2,6-phenylazo)-1,3-dihydroxybenzene/Acid Orange 24;
Acid Black 1 (sodium salt of 1-amino-2-(4′-nitrophenylazo)-7-phenylazo-8-hydroxynaphthalene-3,6-disulphonic acid);
(4-((4-methylphenyl)sulphonyloxy)phenylazo)2,2′-dimethyl-4-((2-hydroxy-5,8-disulphonato)naphthylazo)biphenyl/Acid Red 111;
Food Black 2;
1-(4′-sulphonatophenylazo)-4-((2″-hydroxy-3″-acetylamino-6″,8″-disulphonato)naphthylazo)-6-sulphonatonaphthalene (tetrasodium salt)/Food Black 1;
4-β-hydroxyethylamino-3-nitro benzenesulphonic acid;
Acid Blue 9;
(5′,6′ or 7′)-sulphonato-6′-methylquinoline-2,2′,Δ-1,3-indanedione/Acid Yellow 3;
sodium salt of 4-hydroxy-3((2-methoxyphenyl)-azo)-1-naphthalenesulphonic acid/Acid Red 4;
2-piperidino 5-nitrobenzenesulphonic acid;
2-(4′-N,N-(2″-hydroxyethyl)amino-2′-nitro)anilineethanesulphonic acid;
4-β-hydroxyethylamino-3-nitrobenzenesulphonic acid;
Acid Violet 49;
Acid Blue 7;
Acid Blue 156;
Acid Blue 317;
Acid Blue 62.

Most of these dyes are described in particular in the Color Index published by The Society of Dyers and Colorists, P.O.Box 244, Perkin House, 82 Grattant Road, Bradford, Yorkshire, BD1 2JBN England.

The basic dyes that can be used in the context of this invention can be chosen from nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylamethane-derived dyes and basic natural dyes, and mixtures thereof.

Among the direct dyes that can be used according to the invention, mention may be made of the cationic dyes described in Patent Applications WO 95/15144, WO 95/01772, EP 714954, FR 2 822 696, FR 2 825 702, FR 2 825 625, FR 2 822 698, FR 2 822 693, FR 2 822 694, FR 2 829 926, FR 2 807 650, WO 02/078660, WO 02/100834, WO 02/100369, FR 2 844 269.

Among these compounds, mention may most particularly be made of the following dyes:
1,3-dimethyl-2-[[4-(dimethylamino)phenyl]azo]-1H-imidazolium chloride;
1,3-dimethyl-2-[(4-aminophenyl)azo]-1H-imidazolium chloride;
1-methyl-4-[(methylphenylhydrazono)methyl]-pyridinium methyl sulphate;
Basic Red 22;
Basic Red 76;
Basic Yellow 57;
Basic Brown 16;
Basic Blue 41;
Basic Blue 67;
Basic Brown 1;
Basic Brown 4;
Basic Red 18;
Basic Red 46;
Basic Red 104;
Basic Red 118;
Basic Violet 35;
Basic Yellow 45;
Basic Yellow 67;
Basic Red 14;
Basic Yellow 13;
Basic Yellow 29;
Basic Brown 17;
Basic Blue 22;
Basic Blue 99;
1-N-methylmorpholiniumpropylamino-4-hydroxyanthraquinone;
Basic Blue 17;
Basic Red 2;
Basic Green 1;
Basic Violet 3;
Basic Violet 14;
Basic Blue 7;
Basic Blue 26.

It is understood that the ionic block of the block copolymer will be an anionic block, when the dye is a cationic dye (or basic direct dye), and that the ionic block of the block copolymer will be a cationic block, when the dye is an anionic dye (or acidic direct dye).

According to the invention, it is specified that the expression “water-soluble nonionic block” is generally intended to mean a polymer block comprising at least 80 mol % of at least one water-soluble monomer and, optionally, up to 20 mol % of one or more water-insoluble monomers, this or these water-insoluble monomer(s) being distributed randomly within the water-soluble block. This proportion of water-insoluble monomers is preferably at most equal to 10 mol %, and even better still less than or equal to 5 mol %.

It is specified that the expression “(polymeric or monomeric) water-soluble compound” is generally intended to mean a compound which, when introduced into water at 25° C., and if need be neutralized, at a concentration by weight equal to 0.1%, makes it possible to obtain a macroscopically homogeneous and transparent solution or suspension, i.e. having a light transmittance value, at a wavelength equal to 500 nm, through a sample 1 cm thick, of at least 70%, preferably at least 80%.

Preferably, the block copolymers used in the compositions of the invention are devoid of saccharide units.

The anionic blocks may result advantageously from the polymerization of one or more monomers chosen from monomers bearing at least one carboxylic acid function, monomers bearing at least one sulphonic acid function, monomers bearing at least one phosphonic acid function, and mixtures thereof.

Monomers bearing one or more suitable carboxylic function(s) can be chosen from (meth)acrylic acid, aspartic acid, 1,4-phenylenediacrylic acid, citraconic acid, trans-cinnamic acid, 4-hydroxy-3-methoxycinnamic acid, p-hydroxycinnamic acid, trans-glutaconic acid, glutamic acid, itaconic acid, linoleic acid, linolenic acid, maleic acid, methylfumaric acid, trans-hex-3-enedioic acid, trans-trans muconic acid, oleic acid, ricinoleic acid, trans-traumatic acid, vinylbenzoic acid and vinylglycolic acid.

Monomers bearing one or more suitable sulphonic function(s) can be chosen from 2-propene-1-sulphonic acid, acrylamidopropanesulphonic acid, 4-styrenesulphonic acid and vinylsulphonic acid.

Monomers bearing one or more suitable phosphonic function(s) may be vinylsulphonic acid.

It is understood that the monomers listed above can exist in the form of salts, such as sodium salts, potassium salts or ammonium salts.

According to the invention, the anionic block of the invention is advantageously a block resulting from the polymerization of methacrylic acid, optionally in the form of salts, and even more preferably of acrylic acid, optionally in the form of salts.

The cationic blocks can be blocks of polyamino acids (such as polylysine), blocks of alkanolamine esters of polymethacrylic acid (such as poly(dimethylammonioethyl methacrylate)), blocks of polyamines (such as polyspermine or polyethyleneimine) or blocks of polyvinylpyridine.

The water-soluble nonionic blocks as mentioned above comprise a large proportion of water-soluble nonionic monomers and, optionally, hydrophobic nonionic monomers in small proportions.

The water-soluble nonionic monomers can be chosen from acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolysed), ethylene oxide, hydroxyethyl acrylate, hydroxyethyl methacrylate and hydroxypropyl methacrylate.

The water-insoluble nonionic monomers can be chosen from vinylaromatic monomers which may or may not be alkylated, such as styrene and alkyl styrenes, for instance 4-butylstyrene, α-methylstyrene and vinyltoluene; dienes such as butadiene and 1,3-hexadiene, and alkyl dienes such as isoprene and dimethylbutadiene; chloroprene; C₁₋₁₀ alkyl, C₆₋₁₀ aryl or C₆₋₁₀ aralkyl acrylates, for instance methyl, ethyl, n-butyl, 2-ethylhexyl, tert-butyl, isobornyl, phenyl or benzyl (meth)acrylates; vinyl acetate; vinyl ethers of formula CH₂═CH—CH₂—O—R in which R represents a C₁₋₆ alkyl group; acrylonitrile; vinyl chloride; vinylidene chloride; caprolactone; ethylene; propylene; vinyl monomers that are fluorinated or that contain a perfluoro chain, such as fluoroalkyl acrylates or methacrylates, or alkyl α-fluoroacrylates.

Preferably, the water-insoluble monomers are chosen from aromatic vinyl compounds such as styrene, 4-butylstyrene, α-methylstyrene and vinyltoluene, styrene being particularly preferred.

The number-average molecular mass of each block of the block copolymer preferably ranges from 500 to 10⁶, preferably from 800 to 500 000.

Specific copolymers that can be used in the context of the invention are poly(acryloxyethyltrimethylammonium methylsulphate-b-acrylamide), which will be used with an anionic dye, and poly(sodium acrylate-b-acrylamide), which will be used with a cationic dye.

The block copolymers of the invention can be prepared by the synthetic processes conventionally used to obtain block polymers. Mention may, for example, be made of anionic polymerization and controlled radical polymerization (see “New Method of Polymer Synthesis”, Blackie Academic & Professional, London, 1995, Volume 2, page 1, or Trends Polym. Sci. 4, page 183 (1996) by C. J. Hawker) which can be carried out according to various processes such as, for example, atom transfer radical polymerization (ATRP) (see JACS, 117, page 5614 (1995), by Matyjasezwski et al.) or the method with radicals such as nitroxides (Georges et al., Macromolecules, 1993, 26, 2987).

These processes can also be used to obtain just one of the two types of blocks of the copolymer of the invention, the other block being introduced into the final polymer by means of the initiator used or else by means of a coupling reaction between the two blocks.

The block copolymers of the invention are generally present in an amount ranging from 0.001 to 20% by weight relative to the total weight of the composition, preferably from 0.01 to 10% by weight.

In addition to the presence of a dye and of a block polymer as defined above, the compositions of the invention also comprise a cosmetically acceptable aqueous medium, i.e. a medium that is compatible with keratin fibres, such as the hair.

The composition can also comprise at least one oxidation base. This oxidation base is advantageously chosen from the oxidation bases conventionally used in oxidation dyeing, for example para-phenylenediamines, bisphenylalkylenediamines, para-aminophenols, ortho-aminophenols and heterocyclic bases.

The composition can similarly contain one or more couplers conventionally used for dyeing keratin fibres. Among these couplers, mention may in particular be made of meta-phenylendiamines, meta-aminophenols, meta-diphenols, naphthalenic couplers and heterocyclic couplers.

Generally, the content of oxidation base, and of coupler, when these compounds are present, is between 0.001 and 10% by weight of the total weight of the dye composition, and more preferably from 0.005 to 6% by weight.

In general, the addition salts with an acid that can be used are in particular chosen from hydrochlorides, hydrobromides, sulphates, citrates, succinates, tartrates, lactates, tosylates, benzenesulphonates, phosphates and acetates.

The cosmetically acceptable aqueous medium generally consists of water or of a mixture of water and of at least one organic solvent chosen from C₁-C₄ lower alcohols, such as ethanol, isopropanol, tert-butanol or n-butanol; polyols such as glycerol, propylene glycol and polyethylene glycols.

The pH of the compositions according to the invention is generally between 4 and 11.

The pH can be adjusted to the desired value by means of acidifying or basifying agents normally used in the dyeing of keratin fibres, alternatively using conventional buffer systems.

Among the acidifying agents, mention may, by way of example, be made of inorganic or organic acids such as hydrochloric acid, orthophosphoric acid or sulphuric acid, or carboxylic acids such as acetic acid, tartaric acid, citric acid, lactic acid or sulphonic acids.

Among the basifying agents, mention may, by way of example, be made of aqueous ammonia, alkali metal carbonates, alkanolamines such as mono-, di and triethanolamines and derivatives thereof, sodium hydroxide or potassium hydroxide and the compounds of formula below: 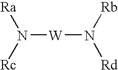
in which W is a propylene residue optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The composition according to the invention may also comprise at least one oxidizing agent. The presence of such an agent may in particular be useful for obtaining various shades of colour from a given dye.

The oxidizing agents that can be used are, for example, hydrogen peroxide, urea peroxide, alkali metal bromates, persalts such as perborates and persulphates, peracids and oxidase enzymes, among which mention may be made of peroxidases, 2-electron oxidoreductases such as uricases, and 4-electron oxygenases such as laccases. Hydrogen peroxide is particularly preferable.

The composition according to the invention may also comprise additives chosen from anionic, nonionic or amphoteric surfactants, anionic or nonionic film-forming polymers that are different from the block polymers described above, cationic or amphoteric conditioning polymers, volatile or nonvolatile, linear, branched or cyclic silicones that may or may not be organomodified, associative or nonassociative polymer thickeners, nonpolymer thickeners, pearlescent agents, opacifiers, dyes or pigments, fragrances, mineral, plant and/or synthetic oils, waxes, vitamins, UV-screening agents, free-radical scavengers, anti-dandruff agents, preserving agents, pH stabilizers and solvents, and mixtures thereof.

Those skilled in the art will take care to choose the optional additives and the amount thereof in such a way that they do not harm the properties of the compositions of the present invention.

These additives are generally present in the composition according to the invention in an amount ranging from 0 to 20% by weight relative to the total weight of the composition.

These compositions may constitute direct dyeing compositions, i.e. that do not need to be revealed with revealing agents such as oxidizing agents.

Thus, another subject of the present invention is a process for the direct dyeing of keratin fibres, comprising the application of at least one dyeing composition as defined above to dry or wet fibres, and then the observation of a leave-in time, optionally followed by rinsing of the fibres.

The leave-in time is generally between a few seconds and 30 minutes, preferably between 3 and 15 minutes.

The temperature at which the composition is left to act is, in general, between 15 and 220° C., more particularly between 15 and 80° C., preferably between 15 and 40° C.

The compositions of the invention can also be compositions to be mixed with oxidation dyeing compositions in order to modulate the dyeing properties thereof or to be an integral part of an oxidation dyeing composition.

The compositions according to the invention, even free of an oxidation dye (oxidation base/coupler), can also be used in the presence of an oxidizing agent.

In this case, the composition according to the invention is applied to the fibres in the presence of an oxidizing agent that can either be mixed with the composition according to the invention (so as to obtain a ready-to-use composition), or applied directly to the keratin fibre.

Conventionally, the oxidizing agent is chosen from the compounds previously described. The content of oxidizing agent is generally between 1 and 40% by weight, relative to the weight of the ready-to-use composition, preferably between 1 and 20% by weight.

In general, the composition comprising the oxidizing agent that is used is an aqueous composition and can be in the form of a solution or alternatively of an emulsion.

Usually, the composition free of oxidizing agent is mixed with approximately 0.5 to 10 equivalents by weight of the oxidizing composition.

It should be noted that the pH of the ready-to-use composition is more particularly between 4 and 12, preferably between 7 and 11.5.

The pH of the composition can be regulated by means of a basifying or acidifying agent, in particular chosen from those previously mentioned in the context of the description according to the invention.

The composition in the presence of the oxidizing agent is therefore applied to dry or wet fibres for a leave-in time sufficient to obtain the desired coloration.

The leave-in time and the temperature at which the composition is left to act are of the same order as those of the process used in the absence of an oxidizing agent.

At the end of the leave-in time, the composition is removed by rinsing with water, optionally followed by washing with a shampoo and rinsing, and then optionally drying.

Finally, the invention relates to the use of a block copolymer as defined above, for increasing the solubility in aqueous medium of dyes as defined above, and the stability in oxidizing and reducing medium, and for improving unison on keratin fibres.

The invention will now be described with reference to the examples that follow, given by way of nonlimiting illustration.

### DETAILED DISCLOSURE OF SPECIFIC EMBODIMENTS

### EXAMPLE 1

In this example, locks of permanent-waved white hair were dyed with the dye (Acid Red 18) used at the concentration of 0.6% in water at pH=9 in the presence or absence of 1%, with respect to active material, of a poly(acryloxyethyltrimethylammonium methylsulphate-b-acrylamide) diblock copolymer, in which each block has, respectively, a molecular mass of 11 000 and 30 000 (called P1 in the table below).

The table below illustrates perfectly the contribution of the diblock copolymer defined above in a basic medium in improving the uptake of the acidic dye.

The locks are read by means of Minolta CM3600d colorimeter in the C.I.E. L*a*b* system (specular components included, illuminant D65, angle 100°).

The lower the value of L*, the more intense the colour.

| Treatment | L* |
|---|---|
| Non-dyed permanent-waved white hair | 60.5 |
| Acid Red 18 at 0.6%, pH = 9 | 60.1 |
| P1 + Acid Red 18, pH = 9 | 51.8 |

It was therefore noted that the lock dyed using the composition according to the invention is significantly more coloured that the control lock and the lock comprising only the acidic dye.

In addition, the chromaticity of the lock obtained using the composition according to the invention is significantly greater than that obtained using the acidic dye alone.

### EXAMPLE 2

In this example, trials intended to demonstrate the protective effect of the block polymers in accordance with the invention with respect to dyes that are unstable in an alkaline oxidizing medium were carried out.

The dye tested is Basic Red 76.

In a first trial, an ammoniacal solution at 0.04% by weight of dye at pH=9 was added to a test tube and the solution was left to stand for 1 hour.

In a second trial, an ammoniacal solution at 0.04% by weight of dye at pH=9 was added to a test tube in the presence of a poly(sodium acrylate-b-acrylamide) diblock copolymer, in which each block has, respectively, a number-average molecular mass of 5000 and 30 000, and in the presence of aqueous hydrogen peroxide at 3%, and the mixture is left to stand for 1 hour.

For the first trial, a degradation of the dye is noted after one hour, reflected by a visible loss of coloration of the solution.

For the second trial, the coloration of the solution remains stable even after standing for 1 hour, which attests to the stability of the dye in a basic oxidizing medium when it is placed in the presence of a block polymer in accordance with the invention.

### EXAMPLE 3

In this example, trials intended to demonstrate the increase in solubility of dyes difficult to solubilize in an aqueous medium, due to the presence of block polymers in accordance with the invention, were carried out.

The dye tested is Astrazon Orange R (or Basic Orange 22).

In a first trial, an aqueous solution comprising 0.04% of dye is added to a test tube. This solution exhibits a cloudy appearance, thus attesting to the poor solubility of this dye in an aqueous medium.

In a second trial, a solution comprising 0.04% of dye, followed by 0.05% by weight of a poly(sodium acrylate-b-acrylamide) diblock copolymer (copolymer of Example 2), are added to a test tube. The resulting solution is a clear solution. This thus attests to the ability of the block polymers in accordance with the invention to increase the solubility of dyes that are difficult to solubilize in an aqueous medium.

What is claimed is:

1. Composition for dyeing keratin fibres, comprising a physiologically acceptable aqueous medium, comprising:
at least one dye bearing at least one ionic group, said dye being present in an amount ranging from 0.001 to 5% by weight relative to the total weight of the composition;
at least one block copolymer comprising at least one water-soluble nonionic block and at least one ionic block exhibiting charges opposite to that of the ionic charge borne by the dye.

2. Dyeing composition according to claim 1, in which the dye is chosen from acidic direct dyes, when the ionic block is a cationic block.

3. Dyeing composition according to claim 2, in which the acidic direct dyes contain at least one COOH, SO₃H, PO₃H, or PO₄H₂ group, it being possible for said groups to exist in the form of salts.

4. Dyeing composition according to claim 2 or 3, in which the acidic direct dyes are chosen from acidic nitro dyes, acidic azo dyes, acidic azine dyes, acidic triarylmethane dyes, acidic quinone dyes, acidic indoamine dyes and acidic natural dyes, and mixtures thereof.

5. Dyeing composition according to claim 1, in which the dye is chosen from basic direct dyes, when the ionic block is an anionic block.

6. Dyeing composition according to claim 5, in which the basic direct dyes are chosen from nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylamethane-derived dyes and basic natural dyes, and mixtures thereof.

7. Dyeing composition according to claim 1, in which the water-soluble nonionic block comprises at least 80 mol % of at least one water-soluble nonionic monomer.

8. Dyeing composition according to claim 1, in which the water-soluble nonionic block comprises up to 20 mol % of one or more water-insoluble monomers, this or these water-insoluble monomer(s) being distributed randomly within the water-soluble nonionic block.

9. Dyeing composition according to claim 7, in which the water-soluble nonionic monomer is chosen from the group consisting of acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinyl-formamide, N-vinyllactams comprising a cyclic group of 4 to 9 carbon atoms, vinyl alcohol (copolymerized in the form of vinyl acetate and then hydrolysed), ethylene oxide, hydroxyethyl acrylate, hydroxyethyl methacrylate and hydroxypropyl methacrylate.

10. Dyeing composition according to claim 8, in which the water-insoluble monomer is chosen from the group consisting of vinylaromatic monomers which may or may not be alkylated; dienes which may or may not be alkylated; chloroprene; C₁₋₁₀ alkyl, C₆₋₁₀ aryl or C₆₋₁₀ aralkyl acrylates; vinyl acetate; vinyl ethers of formula CH₂═CH—CH₂—O—R in which R represents a C₁₋₆ alkyl group; acrylonitrile; vinyl chloride; vinylidene chloride; caprolactone; ethylene; propylene; vinyl monomers that are fluorinated or that contain a perfluoro chain.

11. Dyeing composition according to claim 5, in which the anionic block results from the polymerization of one or more monomers chosen from monomers bearing at least one carboxylic acid function, monomers bearing at least one sulphonic acid function, monomers bearing at least one phosphonic acid function, and mixtures thereof.

12. Dyeing composition according to claim 11, in which the monomers bearing at least one carboxylic acid function are chosen from the group consisting of (meth)acrylic acid, aspartic acid, 1,4-phenylenediacrylic acid, citraconic acid, trans-cinnamic acid, 4-hydroxy-3-methoxycinnamic acid, p-hydroxycinnamic acid, trans-glutaconic acid, glutamic acid, itaconic acid, linoleic acid, linolenic acid, maleic acid, methylfumaric acid, trans-hex-3-enedioic acid, trans-trans muconic acid, oleic acid, ricinoleic acid, trans-traumatic acid, vinylbenzoic acid and vinylglycolic acid.

13. Dyeing composition according to claim 11, in which the monomers bearing at least one sulphonic acid function are chosen from the group consisting of 2-propene-1-sulphonic acid, acrylamidopropanesulphonic acid, 4-styrenesulphonic acid and vinylsulphonic acid.

14. Dyeing composition according to claim 2, in which the cationic block is chosen from the group consisting of blocks of polyamino acids, blocks of alkanolamine esters of poly(methacrylic acid), blocks of polyamines and blocks of polyvinylpyridine.

15. Dyeing composition according to claim 1, in which the block copolymer is present in an amount ranging from 0.001 to 20% by weight relative to the total weight of the composition, preferably from 0.01 to 10% by weight.

16. Dyeing composition according to claim 1, also comprising at least one oxidation base chosen from para-phenylenediamines, bisphenylalkylenediamines, para-aminophenols, ortho-aminophenols and heterocyclic bases.

17. Dyeing composition according to claim 1, also comprising at least one coupler chosen from meta-phenylenediamines, meta-aminophenols, meta-diphenols, naphthalenic couplers and heterocyclic couplers.

18. Dyeing composition according to claim 1, having a pH ranging from 4 to 11.

19. Dyeing composition according to claim 1, also comprising an oxidizing agent.

20. Dyeing composition according to claim 19, in which the oxidizing agent is chosen from hydrogen peroxide, urea peroxide, alkali metal bromates, persalts such as perborates and persulphates, peracids, and oxidase enzymes.

21. Dyeing composition according to claim 1, also comprising at least one additive chosen from anionic, nonionic or amphoteric surfactants, anionic or nonionic film-forming polymers that are different from the block copolymers as defined in claim 1, cationic or amphoteric conditioning polymers, volatile or nonvolatile, linear, branched or cyclic silicones that may or may not be organomodified, associative or nonassociative polymer thickeners, nonpolymer thickeners, pearlescent agents, opacifiers, dyes or pigments, fragrances, mineral, plant and/or synthetic oils, waxes, vitamins, UV-screening agents, free-radical scavengers, anti-dandruff agents, preserving agents, pH stabilizers and solvents, and mixtures thereof.

22. Process for dyeing keratin fibres, comprising the application of at least one dyeing composition as defined in claim 1 to dry or wet fibres, and then the observation of a leave-in time, optionally followed by rinsing of the fibres.

23. Use of a block copolymer as defined in claim 1, for improving the solubility in aqueous medium of a dye as defined in claim 1.

24. Use of a block copolymer as defined in claim 1, for improving the stability in oxidizing and reducing medium of a dye as defined in claim 1.

25. Use of a composition as defined in claim 1, for improving unison of the coloration on keratin fibres.
